(19) United States
(12) Patent Application Publication
(10) Pub. No.: US 2010/0151066 A1
(43) Pub. Date: Jun. 17, 2010

(54) COMPOSITION FOR SUPPRESSING APPETITE, IMPROVING TONE AND MOOD, WITH A NATURAL ANTIDEPRESSANT ACTIVITY AND WITH AN ANTIASTHENIC EFFECT

(76) Inventor: Giulia Federica MERIZZI, Torino (IT)

Correspondence Address: Steinfl & Bruno, 301 N Lake Ave Ste 810, Pasadena, CA, 91101, US

(21) Appl. No.: 12/634,429

(22) Filed: Dec. 9, 2009

Related U.S. Application Data
(63) Continuation-in-part of application No. 12/663,249, filed on Dec. 4, 2009.

(30) Foreign Application Priority Data
Jun. 5, 2007 (IT) TO2007A000391

(51) Int. Cl. A61K 35/74; A61P 3/04; A61P 25/00; A61K 31/405

(52) U.S. Cl. 424/780; 514/419

(57) ABSTRACT

Compositions comprising L-tryptophan, 5-hydroxytryptophan and/or D-Limonene are used for preparing a product suitable for sublingual or nasal administration which is helpful in suppressing appetite in such a manner as to promote weight loss in an individual; such compositions furthermore exhibit an antidepressant activity and an antiasthenic effect which makes them helpful in improving tone and mood and the level of attention in an individual. The compositions preferably comprise phenylethylamine in a synergistic combination which substance is contained in an algal plant extract, preferably of Klamath algae.

### CROSS REFERENCE TO RELATED APPLICATIONS

The present application is a continuation in part of U.S. Ser. No. 12/663,249 filed on Dec. 4, 2009 which is the U.S. national phase of PCT application PCT/IB2008/052154 filed on Jun. 3, 2008, which on its turn claims priority of Italian patent application TO2007A000391 filed on Jun. 5, 2007, each of which is incorporated herein by reference in its entirety.

### FIELD

The present invention relates to pharmaceutical or food supplement compositions or a medical device having an antidepressant activity and antiasthenic effect and to the use thereof in suppressing appetite in such a manner as to promote weight loss and for improving tone and mood.

### BACKGROUND

The problem of hunger in an overweight individual has always been the underlying factor in the failure of any diet and also results in swings in level of mood, nervous tension, a feeling of frustration; this situation does not only occur while the attempt is being made to slim, but also when maintaining weight loss, often resulting in the lost weight being regained.

A substance, serotonin, is present in our central nervous system which acts as a neurotransmitter, namely is produced selectively by a nerve ending subsequent to a specific stimulus. Serotonin has an action on some aspects of mood and sleep and an association has been identified between a deficiency of serotonin and depression. Hunger, satiety, emotional equilibrium and some food-related behaviours are all mediated by serotonin in certain regions of the hypothalamus. Chronic headaches, in particular migraines, are also the result of low serotonin levels.

L-Tryptophan is present in the plasma, both in free form and bound to plasma proteins: however, it is only the free form which is capable of passing through the blood-brain barrier in order to be converted into 5-HTP (5-hydroxytryptophan), which is the precursor of serotonin and is subsequently converted into serotonin.

### SUMMARY

The present invention provides for a method for suppressing appetite, promoting weight loss, and/or improving the level of attention, tone and/or mood in an individual and related compositions and systems.

According to an aspect, the method comprises administering a composition comprising Griffonia simplicifolia, and/or a Klamath algae extract, alone or in combination with D-limonene.

According to an additional aspect, the method comprises administering a composition comprising L-tryptophan, 5-hydroxytryptophan and/or D-limonene.

### BRIEF DESCRIPTION OF THE DRAWINGS

The accompanying drawings, which are incorporated into and constitute part of this specification, illustrate one or more embodiments of the present invention and, together with the detailed description and examples sections, serve to explain the principles and implementations of the invention.

FIG. 1 is a diagram showing the results of the Haber test carried out with a composition according to the invention.

### DETAILED DESCRIPTION

The present invention firstly provides the use of a composition comprising L-tryptophan and/or 5-hydroxytryptophan for preparing a product for sublingual or nasal administration which is helpful in suppressing appetite and in promoting weight loss in an individual.

The present invention also provides the use of the above-stated composition for preparing a sublingually or nasally administrable product which has an antidepressant and antiasthenic activity and is thus helpful in improving not only tone and mood, but also the level of attention and mental energy.

The sublingual route of administration is preferred because it permits faster absorption of the substance tryptophan in free form and then through the blood-brain barrier.

To this end, the product provided by the invention is preferably formulated in liquid form for spray application (oral spray); however, other administration forms, such as for example paper, film or soluble tablets for oral/sublingual application, are envisaged.

Preferably used sources of tryptophan or 5-HTP are plant extracts with an elevated 5-HTP content, especially extracts from leguminous plants and in particular extracts from the African plant Griffonia simplicifolia.

Griffonia simplicifolia extracts of a titrated 5-HTP strength with a 5-HTP content generally of between 15% and 25% are commercially available and may be used for the purposes of the invention.

In a preferred embodiment, the product provided by the invention furthermore comprises a source of phenylethylamine (PEA), preferably composed of an extract of blue-green algae, preferably of a Klamath algae extract. It is known that blue-green microalgae contain a significant quantity of phycocyanins, together with variable amounts of phenylethylamine. In particular, Klamath algae is the only food hitherto known to contain significant quantities of phenylethylamine, an amino acid naturally produced by our brain in states of euphoria and joy which directly assists in raising the level of freely circulating dopamine, so increasing dopaminergic transmission.

In the brain, the action of PEA is based on the fact that it has a greater affinity than does dopamine itself for the mechanism for reuptake of dopamine into presynaptic vesicles. This means that, once it reaches the brain, it is captured by the presynaptic vesicles and occupies the site normally occupied by dopamine. This results in an increase in the level of freely circulating dopamine in the presynaptic terminals and in a greater concentration of diffuse dopamine in the synaptic gaps, so strengthening dopaminergic transmission.

This capability of modulating dopaminergic transmission means that PEA has properties of interest for alleviating depression and attention disorders and for improving concentration and mood. For the purposes of the present invention, extracts of Klamath algae are used in compositions helpful in regulating appetite, preferably in a synergistic combination with L-tryptophan and/or 5-HTP.

The present invention accordingly also provides the use of extracts of blue-green algae, particularly of Klamath algae, preferably combined with L-tryptophan and/or 5-HTP, for preparing a product intended for oral, and in particular sublingual, administration which is helpful in suppressing appetite and in promoting weight loss in an individual.

As has been stated, the preferred embodiment envisages combining L-tryptophan and/or 5-HTP with extracts of Klamath algae. Such extracts are commercially available. In particular, commercial extracts typically containing from 0.5% to 2% by weight of phenylethylamine may be used.

The composition according to the invention may moreover comprise further plant extracts, preferably selected from among extracts of Centella asiatica, guarana, Taraxacum, artichoke, Gingko biloba which do or do not comprise biflavones and mixtures thereof; the above-stated extracts being preferably in phytosomal form (complexed with phospholipids). Preferably the composition also comprises D-limonene, preferably in the amount of from 1 g to 30 g/100 ml of the composition.

In the case of a liquid product usable as a sublingual spray, the formulation comprises the above-stated extracts dissolved in an aqueous vehicle, optionally including a pharmaceutically acceptable solvent.

A formulation according to the invention typically contains, relative to 100 ml of formulation:
L-tryptophan or 5-HTP from 100 to 20000 mg, preferably from 1000 to 5000 mg
phenylethylamine from 5 to 2000 mg, preferably from 10 to 300 mg.

It will be understood that the composition may furthermore contain preservatives, such as for example methyl hydroxybenzoate (non-sodium) or propyl hydroxybenzoate.

The compositions according to the invention may be offered for sale as a drug, food supplement or medical device.

### EXAMPLES

The usefulness of the above-described compositions in suppressing appetite was tested by means of studies detailed in the following examples.

### Example 1

### Administration of a Composition Comprising Griffonia Simplifolia Extract

The study was carried out using a double blind protocol on two treatment groups each comprising 15 patients. In a first study, the active product used was a product having the following formulation:
30 ml bottle:
Griffonia simplicifolia titrated to 25%: 3 g dry extract
guarana: 350 mg
Centella leaf extract: 0.9 g (3 g per 100 ml)
Taraxacum leaf extract: 0.9 g (3 g per 100 ml)
artichoke leaf extract: 0.75 g (2.5 g per 100 ml)
fructose syrup or sorbitol syrup: 30%
purified water: q.s.
preservatives: methyl hydroxybenzoate (non-sodium) or propyl hydroxybenzoate: 0.1%.

The patients were recruited in a screening visit (time 0) and were randomly assigned to one of the two groups. A first check (time 1) was carried out after a fortnight and a second check (time 2) thirty days from time 0. Over this period, the patients followed a low-calorie diet and kept a diary recording subjective symptoms relating to feelings of hunger, anxiety and to the difficulty of observing the proposed diet. To this end, a visual analogue scale with values from 1 to 10 was used to indicate the intensity of these symptoms.

The product was administered sublingually with three sprays at a time, every three hours, for a maximum of five times per day (h 07:00, 10:00, 13:00, 16:00, 19:00). A selected subgroup was provided who received an evening dose (h 22:00) to control night-time awakening. Each spray administers a dose of 0.3 ml (containing approx. 30 mg of natural extract of Griffonia simplicifolia, titrated to a tryptophan content of 25%), giving an overall daily dose of 450 mg (equivalent to five daily administrations each comprising three sprays); for the above-stated subgroup, the overall daily dose was 540 mg, in six administrations (always three sprays each).

Treatment lasted thirty days and each patient received detailed information at the start of testing on the nature of the study, its duration and the methods used.

Each item of data, recorded on suitable clinical cards and analysed, revealed the following results: the group having taken the product according to the invention did indeed exhibit greater control of the feeling of hunger, with remission of the symptom for up to 2.5 hours from taking (with a mean value of 1.38 hours); the group treated with the placebo did not exhibit any change, except for three test subjects and for a duration of no more than 30 minutes (with a mean value of 19.8 minutes).

In the subgroup of eight test subjects who had reported the problem of night-time awakening associated with hunger, the four who had taken the product according to the invention had all significantly reduced the frequency of awakening after a fortnight's treatment; of the other four, who had taken the placebo, only one reported a reduction in this problem.

As a result of the better control of the feeling of hunger in the group treated with the product, on completion of the study, a greater reduction in body weight was observed relative to the group treated with placebo. Administration of the product had also brought about a significant reduction in body weight in three patients suffering from type 2 diabetes.

### Example 2

### Administration of a Composition Comprising Griffonia Simplifolia and Klamath Extracts

A second study was carried out using the same formulation shown above in Example 1 and additionally containing:
Klamath extract: 2000 mg in 30 ml.

In this case, analysis of the results confirmed the positive outcome shown above with a mean value for remission of the symptom of a feeling of hunger of 3 hours.

In both studies, the test subjects who were taking the active treatment reported a distinct improvement in mood combined with a better level of attention and “mental energy” with an antiasthenic effect resulting from taking the product.

### Example 3

### Administration of a Composition Comprising Griffonia Simplifolia and Klamath Extracts and Further Comprising D-Limonene

A third study was carried out on ten women using the composition above, the one containing Griffonia and Alga Klamath extracts, to which D-limonene was added. This terpene is known for its anti-inflammatory activity on vascular endothelium. This property can be useful for obese people, who often suffer from cardiovascular problems.

The tested composition included 15 g of D-limonene in 100 ml of the above solution and the ten test subjects were instructed to use the spray in the same way as during the previous study.

The protocol of this present study was identical to the previous one; two evaluations were made, the first after a fortnight, the second one after thirty days. Subjects were instructed to follow the same suggestions on the hypocaloric regime as the previous studies, during the 30 days of use of the formulation. They also kept a diary recording their daily feeling of hunger.

This group assuming the formula containing Griffonia, Klamath and D-limonene unexpectedly experienced a reduction of their feeling of hunger lasting up to 4.4 hours (with a mean value of 3.6 hours) after every administration of the product.

Therefore D-limonene combined with Alga Klamath and Griffonia extracts showed an unpredictable and unexpected significantly greater activity in the appetite control, never described before.

### Example 4

### Synergistic Effect of Combined Administration of Different Principles

A controlled trial was designed to verify the possible synergetic effects on the hunger feeling and mood level obtained by the combined administration as a sublingual spray of Klamath algae extract and Griffonia simplicifolia extract.

In the two months comparative study, thirty-nine overweight volunteer women, randomly divided in three groups of thirteen subjects each, took daily for 60 days a dietary supplement containing, as active ingredient, respectively:
GROUP K: Klamath algae extract (5 mg/ml);
GROUP G: Griffonia simplicifolia extract (0.1 g/ml);
GROUP S: Klamath algae (5 mg/ml) and Griffonia simplicifolia extracts (0.1 g/ml).

All these preparations were formulated as sublingual spray. At the same time all the subjects were suggested to observe a simple personalized dietetic regime.

Hunger feeling was evaluated daily by Haber test and mood level by Beck Inventory Scale test. At the end of the inclusion visit (T0) and after sixty days (T 60) the weight of the subjects was measured.

For what it concerns weight and appetite group K didn't show any significant difference between T0 and T 60, while the group G gave a statistically significant improvement of all the parameters. The groups which used the formulation containing both the extracts displayed a higher and very significant improvement of the parameters (weight: −2.7 kg p<0.0001; BMI: −3.5% p<0.0001), making it possible to hypothesize a synergic action between the two extracts.

The details of the study are as follows.

Thirty-nine (39) overweight female patients in reproductive age were recruited for this study after a screening visit. The average age of recruitment was 34±10 years old.

All the patients had a BMI between 25.0 and 35.0 kg/m². None of them was under estroprogestinic therapy.

Exclusion criteria were the presence of combined pathologies like dislipidemic disorders or hyperglycemia (>120 mg/dl), problems with menstrual cycle or other major pathologies (hypertension, cardiac insufficiency, epathic diseases, nephropathies, etc. . . . ).

The treatment with the three plant supplements was combined with a balanced diet purpose-made for each subject. Every patient was instructed to restrict her daily energy intake by a moderate amount, 2508 kJ/d less than daily requirements based on World Health Organization criteria with a regimen that maintained a prudent balance of macronutrients: 25% of energy from fat, 60% of energy from carbohydrates, and 15% of energy from protein.

In this trial the following parameters were evaluated: hunger sensation (Haber test), mood level (Beck test), weight and body mass index (BMI).

All the data were collected at time zero (T0) and at day sixty (T60). Only Haber test was made every day by all the subjects.

T0 and T60 results were analyzed and compared with a t-Student test for paired data. The same analysis was performed between the final results from different groups to understand if they were respectively statistical significant. Haber values were all collected at the end of the study and the average of days' values were calculated. The averages were plotted in a graphic to better understand the trend of the test.

Women involved in the study were randomly divided in the following groups:
Group G: 13 patients treated with the composition reported below, added with 10.0 g of Griffonia simplicifolia extract per 100 ml;
Group K: 13 patients treated with the composition reported below, added with 0.5 g of Klamath algae extract per 100 ml;
Group S: 13 patients treated with the composition reported below, added with 10.0 g of Griffonia simplicifolia and 0.5 g Klamath algae extracts per 100 ml;

The quantity of tryptophan present in 0.5 g of Klamath algae extract is negligible compared to the quantity of tryptophan coming from Griffonia (more than 100 time less).

Dosage and route of administration: The composition was formulated as a sublingual spray. The dosage scheme was 3 sprays at a time, every ⅔ hours, for a maximum of 5 times per day (7 a.m., 10 a.m., 1 p.m., 4 p.m., 7 p.m.).
Galenic form: Sublingual spray.

### Product Composition per 100 ml (Ancillary Extracts Ad Excipients):

Guarana: 1.1 g
Centella leaf extract: 3 g
Taraxacum leaf extract 3 g
Artichoke leaf extract: 2.5 g
Fructose syrup: 30%
Purified water: q.s. to 100 ml
Preservatives: methyl hydroxybenzoate (non sodium) or propyl hydroxybenzoate 0.1%

One patient dropped out of the study in group K and one did in group S, both for personal reasons. No drop out in group G. For this reason data are referred to 37 patients instead of 39 expected.

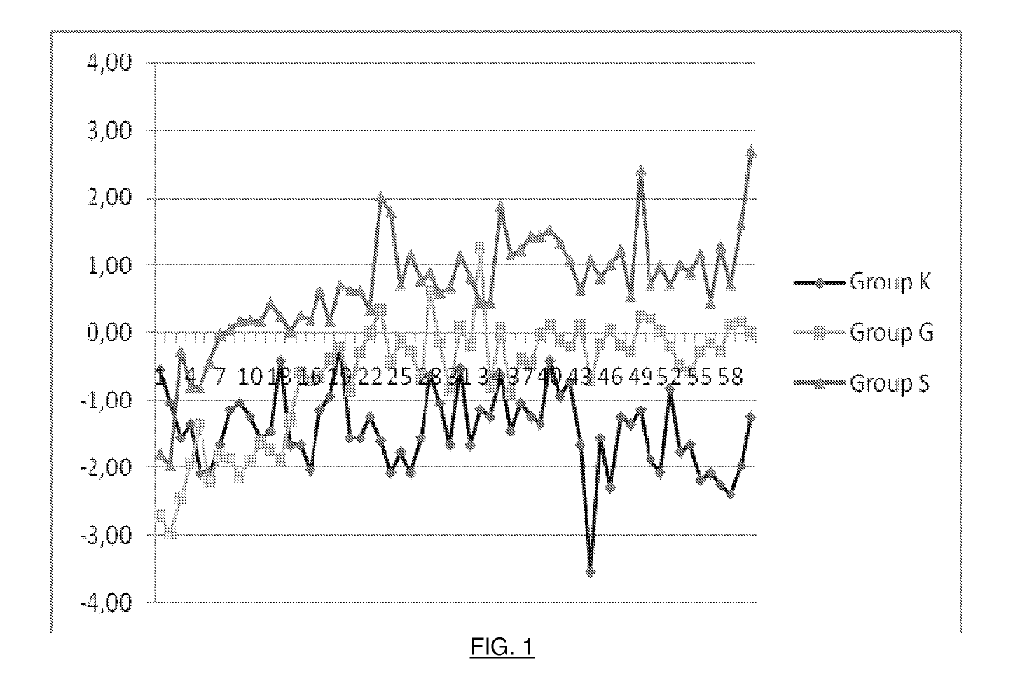

Haber test revealed a significant disparity among the groups for what concerned the feeling of hunger. Group G and group S showed an evident decrease in appetite respectively since day 15^{th} for group G and since day 8^{th} for group S (FIG. 1).

Group K didn't show any significant modification of this parameter for the whole duration of the study, so it seems reasonable to assert that this composition, with a low dosage of Klamath algae alone, doesn't have any effects on hunger feeling (FIG. 1).

Women weight and BMI were also evaluated.

After treatment, measurements showed that there was a significant reduction of body weight in group S, −2.7±0.75 kg (p<0.0001), and in group G, −0.89±0.77 kg (p<0.002) but in this last case of lower entity. The variation in group K was not statistically significant, −0.4±0.67 (n.s.).

Consequently we observed some variations in BMI, that decreased 1.0 unit (3.5%; p<0.0001) in group S and 0.34 unit (1.2%; p<0.002) in group G. Obviously group K did not show any variation (−0.5%; p=n.s.).

Mood level, evaluated by Beck questionnaire (BQ), showed a greater improvement for groups S with statistical relevance (p<0.01). Group G and K showed a statistically significant improvement of lower entity in comparison to group S.

The comparisons between the two groups, G and S, was statistically significant, p<0.0001, with reference to both body weight and BMI reductions.

Tryptophan's inhibitory activity on hypothalamic nervous centre of appetite is well-known and has been widely described in literature. This amino acid is present in relevant quantity in Griffonia Simplicifolia, and in Klamath algae. Moreover this last extract naturally contains phenylethylamine, a physiological molecule present in human body and in food, like chocolate, which is able to increase mood level.

The extracts from the two plants combined together in the same composition and administered by sublingual spray showed a significant synergic efficacy, higher than the one obtained by the addition of the efficacy of the two separate extracts.

In fact, the results obtained in this study underline that the combination of the two plant extracts is more effective than the addition of the results of the two single extracts.

The composition containing both extracts showed to be effective as body weight loss treatment during a restricted dietetic regime mainly due to its activity in decreasing the hunger feeling and by maintaining an appreciable mood level at the same time. This synergic action makes it easier to follow the suggested daily regime and reduce body weight.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the products, methods and system of the present disclosure, examples of appropriate suitable materials and methods are described herein for guidance purpose.

A number of embodiments of the disclosure have been described. Nevertheless, it will be understood that various modifications may be made without departing from the spirit and scope of the present invention. Accordingly, other embodiments are within the scope of the following claims.

1. A method comprising
administering to an individual a composition comprising an extract of Griffonia simplicifolia, and/or a Klamath algae extract, the composition further comprising D-Limonene,
wherein the extract of Griffonia simplicifolia comprises L-tryptophan and/or 5-hydroxytryptophan and
wherein the administering is performed by oral spray and results in at least one of
suppressing appetite, promoting weight loss, and improving the level of attention, tone and/or mood in the individual.

2. The method according to claim 1, wherein said composition comprising the Klamath algae extract comprises phenylethylamine.

3. The method according to claim 1, wherein said composition comprises L-tryptophan and/or 5-hydroxytryptophan in a quantity of 100 mg to 20000 mg/100 ml.

4. The method according to claim 1, wherein said composition comprises phenylethylamine in a quantity of between 5 mg and 2000 mg/100 ml.

5. The method according to claim 3, wherein said composition comprises phenylethylamine in a quantity of between 5 mg and 2000 mg/100 ml.

6. The method according to claim 1, wherein said composition comprises D-limonene in the amount of from 1 g to 30 g/100 ml.

7. The method according to claim 1, wherein said composition furthermore comprises one or more plant extracts selected from the group consisting of guarana extract, artichoke extract, Centella extract, Taraxacum extract, Gingko biloba extract including or not including biflavones, preferably in phytosomal form, and mixtures thereof.

8. The method according to claim 1, wherein said composition comprises L-tryptophan and/or 5-hydroxytryptophan in a quantity of 1000 to 5000 mg/100 ml.

9. The method according to claim 1, wherein said composition comprises phenylethylamine in a quantity of 10 to 300 mg/100 ml.

10. The method according to claim 8, wherein said composition comprises phenylethylamine in a quantity of 10 to 300 mg/100 ml.

11. The method according to claim 10, wherein said composition comprises D-limonene in the amount of from 1 g to 30 g/100 ml.

12. A method comprising
administering to an individual a composition comprising an extract of Griffonia simplicifolia, and/or a Klamath algae extract,
wherein the extract of Griffonia simplicifolia comprises L-tryptophan and/or 5-hydroxytryptophan and
wherein the administering is performed by oral spray and results in at least one of
suppressing appetite, promoting weight loss, and improving the level of attention, tone and/or mood in the individual.

13. The method according to claim 12, wherein said composition comprising the Klamath algae extract comprises phenylethylamine.

14. The method according to claim 12, wherein said composition comprises L-tryptophan and/or 5-hydroxytryptophan in a quantity of 100 mg to 20000 mg/100 ml.

15. The method according to claim 12, wherein said composition comprises phenylethylamine in a quantity of between 5 mg and 2000 mg/100 ml.

16. The method according to claim 14, wherein said composition comprises phenylethylamine in a quantity of between 5 mg and 2000 mg/100 ml.

17. The method according to claim 12, wherein said composition further comprises one or more plant extracts selected from the group consisting of guarana extract, artichoke extract, Centella extract, Taraxacum extract, Gingko biloba extract including or not including biflavones, preferably in phytosomal form, and mixtures thereof.

18. The method according to claim 12, wherein said composition comprises L-tryptophan and/or 5-hydroxytryptophan in a quantity of 1000 to 5000 mg/100 ml.

19. The method according to claim 12, wherein said composition comprises phenylethylamine in a quantity of 10 to 300 mg/100 ml.

20. The method according to claim 18, wherein said composition comprises phenylethylamine in a quantity of 10 to 300 mg/100 ml.
